(19) United States
(12) Patent Application Publication
(10) Pub. No.: US 2024/0382948 A1
(43) Pub. Date: Nov. 21, 2024

(54) MANIFOLDS, SYSTEMS AND METHODS FOR MEASURING CELL FUNCTIONS

(71) Applicant: 3D SYSTEMS, INC., Rock Hill, SC (US)

(72) Inventor: Jordan Miller, Houston, TX (US)

(73) Assignee: 3D SYSTEMS, INC., Rock Hill, SC (US)

(21) Appl. No.: 18/613,776

(22) Filed: Mar. 22, 2024

Related U.S. Application Data
(60) Provisional application No. 63/492,083, filed on Mar. 24, 2023.

(51) Int. Cl. B01L 3/00; B33Y 80/00

(52) U.S. Cl. CPC B01L 3/502; B33Y 80/00; B01L 2200/026; B01L 2300/0681; B01L 2300/0832

(57) ABSTRACT

Various embodiments of the present disclosure disclose manifolds, methods, and systems for measuring cell functions. In various embodiments, a first reservoir storing a first fluid and a second reservoir storing a second fluid may be fluidically connected via a fluid channel. Further, the first fluid and the second fluid may be such that an entropic gradient force causes the first fluid to flow towards a container that is located therebetween and contains cells therein. The cells release cell products into the first fluid flowing in the fluidic channel, which in turn flows into the second reservoir. The cell products can be sampled from the second reservoir and/or from the container to measure the function of the cells.

### CROSS REFERENCE TO RELATED APPLICATIONS

This application claims priority to U.S. provisional patent application Ser. No. 63/492,083 filed Mar. 24, 2023, the entire content of which are incorporated herein by reference and relied upon.

### BACKGROUND

Pre-clinical research and drug development generally rely on testing the behavior of human cells in petri dishes and animal models of human disease to understand the physiology and predict the performance of therapeutic drugs in the human body. The research environment, however, may not adequately represent the complex networked interactions actually taking place in the human body. For example, the cellular environment as well as the testing processes may not accurately reflect the true cellular microenvironment and the actual processes occurring in the human body. As such, improved platforms and techniques that reflect more accurate cellular microenvironment and bodily processes are needed.

### SUMMARY

In various embodiments, a bio-assembly comprising a container, a first reservoir, a second reservoir, a fluidic channel, and a plug is disclosed. In various embodiments, the container is defined within a semi-permeable material; the first reservoir is configured to receive a first fluid; and the second reservoir is configured to receive a second fluid. Further, the fluidic channel fluidically connects the first reservoir and the second reservoir, and the first fluid and the second fluid are such that an entropic gradient force causes the first fluid in the first reservoir to flow towards the second reservoir via the fluidic channel. In various embodiments, the plug is positioned between the second reservoir and the fluidic channel, and is configured to prevent the second fluid from flowing out of the second reservoir via the fluidic channel. In various embodiments, the container is in fluidic communication with the fluid channel and is configured to intake a portion of the first fluid flowing in the fluidic channel from the first reservoir towards the second reservoir and release effluent from the container into the first fluid flowing in the fluidic channel towards the second reservoir

In various embodiments, a method for measuring cell function is disclosed. The method comprises adding a first fluid into a first reservoir of a bio-assembly. Further, the method comprises adding a second fluid into a second reservoir of the bio-assembly. In addition, the method comprises depositing cells into a container of the bio-assembly. In various embodiments, the bio-assembly includes a fluidic channel fluidically connecting the first reservoir and the second reservoir and the first fluid and the second fluid are such that an entropic gradient force causes the first fluid in the first reservoir to flow towards the second reservoir via the fluidic channel. The method further comprises performing a measurement on cell products produced by the cells and released into the first fluid flowing in the fluidic channel towards the second reservoir. In various embodiments, the bio-assembly further includes a plug positioned between the second reservoir and the fluidic channel, the plug configured to prevent the second fluid in the second reservoir from flowing out of the second reservoir via the fluidic channel. In various embodiments, the container is in fluidic communication with the fluidic channel to (i) intake a portion of the first fluid flowing in the fluidic channel from the first reservoir towards the second reservoir; and (ii) release the cell products produced by the cells into the first fluid flowing in the fluidic channel towards the second reservoir.

In various embodiments, a method for determining an optimal therapeutic window for a therapeutic drug is disclosed. The method comprises adding a first fluid including a therapeutic drug to a first reservoir of a bio-assembly. Further, the method comprises adding a second fluid to a second reservoir of the bio-assembly. In various embodiments, the first fluid and the second fluid are such that an entropic gradient force causes the first fluid in the first reservoir to flow towards the second reservoir via a fluidic channel fluidically connecting the first reservoir to the second reservoir. Further, the method comprises depositing cells into a hermetically scalable container of the bio-assembly. Further, the method comprises obtaining a sample volume from the second reservoir. In addition, the method comprises measuring the sample volume to quantify a level of therapeutic drug and a cell product. The method further comprises determining a therapeutic profile for the therapeutic drug based on the level of therapeutic drug and cell product in the sample volume.

In various embodiments, a system for measuring cell function comprising a manifold, a first fluid source, and a second fluid source is disclosed. In various embodiments, the manifold includes a plurality of bio-assemblies, each bio-assembly comprising a container configured to receive cells, a first reservoir configured to receive a first fluid, a second reservoir configured to receive a second fluid, a fluidic channel fluidically connecting the first reservoir to the second reservoir, and a plug positioned between the second reservoir and the fluidic channel and configured to prevent the second fluid in the second reservoir from flowing out of the second reservoir via the fluidic channel. In various embodiments, the first fluid and the second fluid are such that an entropic gradient force causes the first fluid in the first reservoir to flow towards the second reservoir via the fluidic channel. In various embodiments, the first fluid source is in fluidic communication with the first reservoir to provide the first reservoir with the first fluid. In various embodiments, the second fluid source is in fluidic communication with the second reservoir to provide the second reservoir with the second fluid.

In various embodiments, a kit comprising a container defined within a semi-permeable material; a first reservoir containing a first fluid; a second reservoir containing a second fluid; a fluidic channel fluidically connecting the first reservoir and the second reservoir; and a plug positioned between the second reservoir and the fluidic channel is disclosed. In various embodiments, the first fluid and the second fluid are such that an entropic gradient force causes the first fluid in the first reservoir to flow towards the second reservoir via the fluidic channel. Further, the plug is configured to prevent the second fluid from flowing out of the second reservoir via the fluidic channel. In various embodiments, the container is in fluidic communication with the fluid channel and is configured to intake a portion of the first fluid flowing in the fluidic channel from the first reservoir and release cell products produced by cells housed in the container into the first fluid flowing in the fluidic channel towards the second reservoir. Further, equilibrium between the first fluid in the first reservoir and the second fluid in the second reservoir is reached at least a threshold duration after the flow of the first fluid from the first reservoir towards the second reservoir is initiated due to the entropic gradient force.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 is a schematic diagram of an example system for measuring cell function, according to various embodiments.

FIG. 2 is a schematic diagram of an example system with a common fluid reservoir in fluid communication with multiple bio-assemblies for measuring cell function, according to various embodiments.

FIG. 3 is a schematic diagram of an example system with multiple fluid reservoirs in fluid communication with a single container of a bio-assembly for measuring cell function, according to various embodiments.

FIG. 4 is a schematic diagram of an example manifold of multiple bio-assemblies for measuring cell function, according to various embodiments.

FIG. 5 is an example illustration of a bio-assembly for measuring cell function, according to various embodiments.

FIGS. 6A-6B show example illustrations of an application of the example system for measuring cell function, according to various embodiments.

FIG. 7 is a schematic workflow for determining an optimal therapeutic window for a therapeutic drug, according to various embodiments.

FIG. 8 is a flowchart illustrating a method for measuring cell function, according to various embodiments.

FIG. 9 is a flowchart illustrating a method for determining an optimal therapeutic window for a therapeutic drug, according to various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

### I. Overview

In vitro studies have long been standard experimental tools for conducting biological investigations for research, drug development, etc., efforts. For example, to screen therapeutic drugs or compounds for treating human diseases, plastic petri dishes or well plates have been utilized to culture various microorganisms and cells, which can then be monitored overtime to understand the physiology of the microorganisms and cells to identify candidate drugs for further testing. The studies may also be augmented with studies of animal models of the human diseases, mice being the choice as animal models for most biological investigations owing to their relatively low cost and case of handling and use.

The in vitro cellular environment of the petri dishes or well plates, however, may not adequately represent the highly complex and networked tissues of human bodies. Further, despite their popularity in candidate compound screening and other biological investigations, small animal models like mice may not be ideal for such purposes, because they do not approximate human physiology or anatomy adequately enough for a compound that is found to be efficacious in mice to have similar efficaciousness in human beings. The failure of a large number of candidate drugs during Phase II clinical trials can be attributed at least partially to the fact that successful efficacy results in small animal models do not transfer over during human clinical trials.

To address the above noted issues, bioactive platforms or scaffolds (“bio-scaffolds”) mimicking human physiology and anatomy can be designed and constructed to provide biomimetic human tissue models. Besides for compound screening, the bio-scaffolds can be used for other applications such as but not limited to tissue engineering where cells are placed on the bio-scaffolds to facilitate their growth into desired tissues. For example, the bio-scaffolds can include cell-adhesive and cell-degradable materials where cells can adhere, grow, and migrate onto a matrix they can remodel over time by secreting matrix metalloproteinases and depositing their own extracellular matrix.

To allow cells that are placed on the bio-scaffolds to be under perfusion conditions, the bio-scaffolds may include vasculatures, via which fluids may flow. The fluids that may flow into and out of the bio-scaffold can be gases or liquids, such as but not limited to blood, therapeutic drugs, cell culture media, solutions, reagents, and/or the like. Each bio-scaffold may be contained within a bio-assembly that may be in fluidic communication with one or more external reservoirs as well as with the vasculatures of the bio-scaffold such that the bio-scaffold and the one or more external reservoirs may exchange the fluids. In some instances, the bio-assemblies may be arranged into a manifold (e.g., array) of bio-assemblies where the inlets and outlets of each bio-assembly may be in fluidic communication with an inlet reservoir and an outlet reservoir, respectively. Further details on such bio-assemblies and manifolds, and in uses thereof, can be found in Applicant's PCT Application No. PCT/US2021/030668, filed May 4, 2021, titled “Microcosm Bio-Scaffold And Applications Thereof,” and PCT Application No. PCT/IB2022/058885, filed Sep. 22, 2022, titled “Manifolds, Systems And Methods for Conducting Biological Studies under Flow,” both of which are incorporated herein by reference in their entirety.

In various embodiments, the bio-assemblies may not necessarily include bio-scaffolds, but may include contained spaces in which biological activities may occur. For example, the contained spaces may be defined by containers configured to receive cells and provide the received cells an environment in which the cells may undergo biological processes (e.g., which can be monitored or studied to test cell functions). A container of a bio-assembly can be semi-permeable and may be connected to a first reservoir and a second reservoir via a fluid channel that is in fluidic communication with the container such that fluids may be exchanged between the container, the first reservoir, and the second reservoir, thereby allowing the cells contained within the container to be under perfusion conditions. For example, the reservoirs may be connected to external pumps such as but not limited to syringe pumps, peristaltic pumps, pneumatic pumps, gravity driven flow pumps, etc., that are configured to pump perfusable media to facilitate the flow of fluids via the fluid connection.

In various embodiments, an “on-plate” pump, i.e., a pump that is disposed on a bio-assembly, may be used to pump fluids between the first reservoir of the assembly and the second reservoir of the assembly via the fluid channel. Because the container is in fluidic communication with the fluidic channel, the container intakes fluids flowing via the fluid channel, while in some instances discharging other fluids and effluent (e.g., cell products, waste, etc.) into the fluid channel. In some instances, the bio-assembly may not be connected or coupled to an external pump (e.g., such as any one of the afore-mentioned external pumps), but rather the pumping of fluids may be accomplished by on-plate pumps only. In some instances, that the on-plate pumps may be different from any of the afore-mentioned external pumps. For example, an on-plate pump may not be any one of a syringe pump, a peristaltic pump, a pneumatic pump, or any other similar pump.

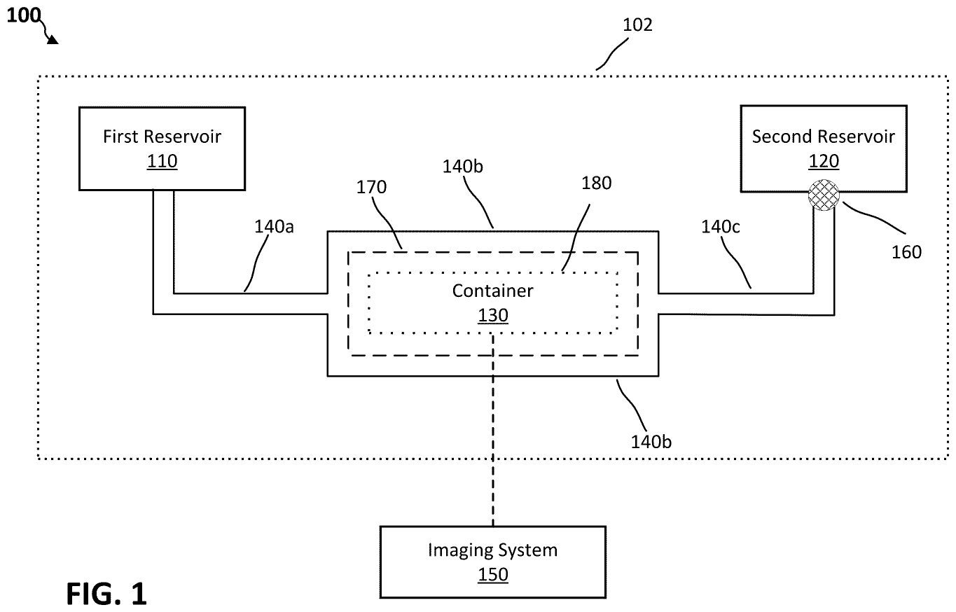

In various embodiments, the on-plate pump may not be a physical pump but rather may be the result of a pressure/force gradient or differential developed between the fluid in one of the first reservoir and the second reservoir, and the fluid in the other one of the first reservoir and the second reservoir. The pressure or force gradient can cause fluids to flow between the first reservoir and the second reservoir via the fluid channel, facilitating the exchange of fluids and cell products between the container and the fluid channel as mentioned above. In some embodiments, the pressure or force gradient can be an entropic gradient force developed between the first fluid and the second fluid due to the entire system's (e.g., including the bio-assembly, the first reservoir and the second reservoir) statistical tendency to increase its entropy. For example, the entropic gradient force can be caused by the hypertonicity of one of the first fluid or the second fluid with respect to the other fluid. That is, for example, when the second fluid is hypertonic with respect to the first fluid, the hypertonicity of the second fluid with respect to the first fluid may develop an entropic gradient force (e.g., osmotic force) that causes the first fluid to flow out of the first reservoir and flow towards the second reservoir via the fluidic channel connecting the first reservoir and the second reservoir. The pressure or force gradient can also be caused by potential energy (e.g., gravitational potential energy) differences between the first fluid and the second fluid. Various embodiments, configurations, and implementations of bio-assemblies, or manifolds thereof, incorporating pressure gradient pumps are described in further detail with respect to FIGS. 1-9.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification.

### II. Example Descriptions of Terms

As used herein the specification, “a” or “an” may mean one or more. As used herein in the claim(s), when used in conjunction with the word “comprising,” the words “a” or “an” may mean one or more than one. Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The use of the term “or” in the claims is used to mean “and/or” unless explicitly indicated to refer to alternatives only or the alternatives are mutually exclusive, although the disclosure supports a definition that refers to only alternatives and “and/or.” For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment. As used herein “another” may mean at least a second or more.

The term “ones” means more than one.

As used herein, the term “plurality” can be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination

As used herein, the term “about” refers to include the usual error range for the respective value readily known. Reference to “about” a value or parameter herein includes (and describes) embodiments that are directed to that value or parameter per se. For example, description referring to “about X” includes description of “X”. In some embodiments, “about” may refer to ±15%, ±10%, ±5%, or ±1% as understood by a person of skill in the art.

As used herein, the term “fluid communication” between a first component and a second component refers to a flow of fluid via a mechanism such as a channel, vascular component, tube, pipe, or network thereof, etc., that extends or exists between the first component and the second component such that fluid may be transported between the first component and the second component via the mechanism with little or no loss or leak. In some cases, the fluid communication may be unidirectional or bidirectional. In some embodiments, fluid communication may also refer to flow of fluid via a membrane or a wall that is separating the first component from the second component.

While the present teachings are described in conjunction with various embodiments, it is not intended that the present teachings be limited to such various embodiments. On the contrary, the present teachings encompass various alternatives, modifications, and equivalents, as will be appreciated by those of skill in the art.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

### III. Systems for Measuring Cell Functions

FIG. 1 shows a schematic diagram of a system 100 for measuring cell function, according to various embodiments. In various embodiments, the system 100 for measuring cell function includes a bio-assembly 102 that comprises a first reservoir 110, a second reservoir 120, a container 130, a plug 160, and a fluid channel including a first fluid channel section 140a, a second fluid channel section 140b, and a third fluid channel section 140c (collectively referred to as fluid channel 140). In various embodiments, the system 100 can also include an imaging system 150. In some instances, the system 100 may not include an imaging system 150 (e.g., when cell product measurements are performed offline by sampling cell products from the container 130 or the second reservoir 120 as discussed below).

In various embodiments, the first reservoir 110 may be configured to receive and store a first fluid, and further allow the first fluid to flow out of the first reservoir 110 when the first fluid is under pressure. The first reservoir 110 may be in fluid communication with the fluid channel 140 (e.g., via the first fluid channel section 140a) so as to allow the first fluid contained therein to flow out of the first reservoir 110 when the first fluid is under pressure. The first reservoir 110 may be shaped and sized to be suitable to receive the first fluid when the first fluid is added into the first reservoir 110 and also allow the flow of the first fluid out of the first reservoir 110 under pressure (e.g., the first reservoir 110 can have a generally conical shape with a wider base shaped and sized to receive the fluid from a pipette and a narrower base shaped to facilitate the flow of the first fluid into the first fluid channel section 140a when the first fluid is under pressure in the first reservoir 110).

In various embodiments, the first reservoir 110 may be manufactured by applying an additive manufacturing technique to any suitable material such as but not limited to a resin, a polycarbonate, an acrylic, a glass, a plastic, a combination thereof, and/or the like. The additive manufacturing technique may include one or more of a computed axial lithography (CAL) technique, 3D printing technique such as but not limited to injection molding technique, rapid casting, sacrificial molding, and/or the like. For example, one or more of the above suitable materials may be injected molded or 3D printed to construct a first reservoir 110 having desired configurations (e.g., shape, size, etc.). For instance, the firstreservoir 110 can be formed via casting around a pattern, such as a structure, which can then be removed mechanically, chemically, and/or by light-induced degradation, followed by patterning one or more pieces and then bonding the pieces together.

In various embodiments, the second reservoir 120 may be configured to receive and store a second fluid. The second reservoir 120 may be in fluid communication with the fluid channel 140 (e.g., with the third fluid channel section 140c) so as to allow fluid flowing in the fluid channel 140 (i.e., the third fluid channel section 140c) to flow into the second reservoir 120. The second reservoir 120 may be shaped and sized to be suitable to receive the second fluid when the second fluid is added into the second reservoir 120 (e.g., the second reservoir 120 can have a generally conical shape with a wider base shaped and sized to receive the second fluid from a pipette).

In various embodiments, the second reservoir 120 may be manufactured by applying an additive manufacturing technique to any suitable material such as but not limited to a resin, a polycarbonate, an acrylic, a glass, a plastic, a combination thereof, and/or the like. The additive manufacturing technique may include one or more of a CAL technique, a 3D printing technique such as but not limited to injection molding technique, rapid casting, sacrificial molding, and/or the like. For example, one or more of the afore-mentioned suitable materials may be injected molded or 3D printed to construct a second reservoir 120 having desired configurations (e.g., shape, size, etc.). For instance, the second reservoir 120 can be formed via casting around a pattern, such as a structure, which can then be removed mechanically, chemically, and/or by light-induced degradation, followed by patterning one or more pieces and then bonding the pieces together.

In various embodiments, the plug 160 may be configured to regulate the flow of fluid between the second reservoir 120 and the fluid channel 140 (i.e., the third fluid channel section 140c) when the plug 160 is disposed or positioned therebetween. In some instances, the plug 160 may be a separate component that is configured to couple with one or both of the second reservoir 120 and the fluid channel 140 at the position where the two meet. In some instances, the plug 160 can be a part of either or both of the second reservoir 120 and the fluid channel 140 (e.g., the plug 160 can be the component that joins the second reservoir 120 to the fluid channel 140).

In various embodiments, the plug 160 may regulate the flow of fluids between the second reservoir 120 and the fluid channel 140 by preventing the flow of fluids out of the second reservoir 120 while allowing fluids inflow from the fluid channel 140. For example, the plug 160 can be a semi-permeable membrane that prevents or at least limits the second fluid of the second reservoir 120 from flowing out of the second reservoir 120 and into the fluid channel 140, while allowing the fluids in the third fluid channel section 140c to flow into the second reservoir 120. In some instances, the plug 160 may be configured to prevent the outflow of fluids from the second reservoir 120 based on the molecular weight of the fluids. For example, the plug 160 may be configured to allow second fluids of the second reservoir 120 with molecular weight less than a threshold molecular weight to flow out of the second reservoir 120 while preventing outlet-side fluids with molecular weight no less than the threshold molecular weight from flowing out of the outlet reservoir 120. That is, the plug 160 may be viewed as a filter membrane that keeps in fluids in the second reservoir 120 with molecular weight no less than the threshold molecular weight from flowing out of the second reservoir 120 (e.g., while allowing first fluid from first reservoir 110 and effluent such as cell byproducts from the container 130 flow into the second reservoir 120, as well as allow second fluids in the second reservoir 120 with molecular weight less than the threshold molecular weight flow out of the second reservoir 120). In some examples, the threshold molecular weight is about 1 kDalton (kDa), 3 kDa, 5 kDa, 8 kDa, 10 kDa, 15 kDa, 25 kDa, etc.

In some instances, the plug 160 may be configured to filter fluids exiting the second reservoir 120 as discussed above, but not filter fluids that are entering the second reservoir 120. That is, the plug 160 may be a uni-directional filter. For example, the plug 160 may be configured to allow all fluids in the fluid connection 140 (i.e., the third fluid connection section 140c) to flow into the outlet reservoir 120. For example, the plug 160 may be configured to allow the first fluid of the first reservoir 110 that has flown into the fluid channel 140 from entering the second reservoir 120 without regards to the molecular weight of the first fluid. Further, the plug 160 may be configured to allow effluent flowing out of the container such as but no limited to cell byproducts produced and released into the fluid channel 140 by cells in the container 130 to enter the second reservoir 120 without regard to molecular weight when carried along by the first fluid flowing in the fluid channel 140 towards the second reservoir 160.

In various embodiments, the plug 160 may be made from or include materials such as but not limited to gel, hydrogel (e.g., solid hydrogel), polymerizable hydrogel, collagen methacrylate, silk methacrylate, hyaluronic acid methacrylate, chondroitin sulfate methacrylate, elastin methacrylate, cellulose acrylate, dextran methacrylate, heparin methacrylate, N-isopropyl acrylamide (NIPAAm) methacrylate, Chitosan methacrylate, polyethylene glycol norbornene, polyethylene glycol dithiol, thiolated gelatin, thiolated chitosan, thiolated silk, PEG based peptide conjugates, and/or the like, or any combination thereof.

In various embodiments, the plug 160 may be made by applying an additive manufacturing technique to any of the afore-mentioned suitable materials. The additive manufacturing technique may include one or more of a CAL technique, a 3D printing technique such as but not limited to injection molding technique, rapid casting, sacrificial molding, and/or the like. For example, one or more of the materials may be injected molded or 3D printed to construct the plug 160 having desired configurations (e.g., shape, size, etc.). For instance, the plug 160 can be formed via casting around a pattern, such as a structure, which can then be removed mechanically, chemically, and/or by light-induced degradation, followed by patterning one or more pieces and then bonding the pieces together.

In cases where the plug 160 is a part of the second reservoir 120, the plug 160 can be a hydrogel (e.g., solid hydrogel) that is 3D printed or otherwise additively manufactured on the second reservoir 120, which may be made from, for example, plastics. That is, for instance, the second reservoir 120 and the plug 160 may be constructed together using an additive manufacturing technique where the former is made from a plastic material while the latter is a solid hydrogel 3D printed on the plastic.

In various embodiments, the first fluid of the first reservoir 110 and the second fluid of the second reservoir 120 may be selected such that when the first fluid and the second fluid are added into their respective reservoirs, an entropic gradient force develops that causes a fluid in one of the first reservoir 110 or second reservoir 120 to flow to the other reservoir. For example, the entropic gradient force can cause the first fluid in the first reservoir 110 to flow out of the first reservoir 110 via the first fluid channel section 140a to flow towards the second reservoir 120 via the fluidic channel 140. For instance, the amount and/or types of the first fluid and the second fluid can be such that the system 100 that includes the first reservoir 110, the second reservoir 120, the container 130, the fluidic channel 140, etc., may increase its entropy by developing an entropic gradient force that causes the first fluid in the first reservoir 110 to flow towards the second reservoir 120 (e.g., in accordance with Bernoulli's principle or equation).

In various embodiments, the entropic gradient force can be the result of the hypertonicity of the second fluid with respect to the first fluid such that an osmotic force causes the first fluid to flow out of the first reservoir 110 and into the first fluid channel section 140a. That is, the hypertonicity of the second fluid with respect to the first fluid, which are in fluid communication with each other via the fluid channel 140, can generate an osmotic pressure or force on the first fluid in the first reservoir 110 to cause the first fluid to flow towards the second reservoir 120 via the fluid channel 140.

Examples of the inlet-side fluid can be or include cell culture media, therapeutic drugs, bioactive factors, and/or the like. For instance, the cell culture media can include a nutrient, a glucose solution, a serum, an amino acid, an inorganic salt, a vitamin, a combination thereof, and/or the like. The therapeutic drug can include a chemotherapeutic drug, an antibiotic, a combination thereof, and/or the like. The bioactive factor can include a growth factor, an intracellular signaling molecule, a signaling mimetic, a combination thereof, and/or the like.

Examples of the outlet-side fluid can be or include a hypertonic polymer solution, a stabilizing reagent, a combination thereof, and/or the like. For instance, the hypertonic polymer solution can be a polyethylenimine (PEI) solution, and/or the like. As another example, the stabilizing reagent can include a phosphate, an amorphous calcium carbonate, or a combination thereof, and/or the like.

In various embodiments, the container 130 may be configured to receive and store specimens with which one wishes to use the bio-assembly 102 to perform measurements (e.g., for cell functions), undertake experimental investigations, etc. For example, the container 130 may be shaped and sized to facilitate receiving and holding the specimen, such as having a generally conical shape with the wider base shaped and sized to receive the specimen from a pipette and the narrower base shaped and sized to allow the received specimen accumulate. Without limit, the specimen can be biological samples such as cells or cell types that can form one or more layers including endothelial layers, epithelial layers, smooth muscle cell layers, sequentially delivered smooth muscle cell layers and endothelial layers, sequentially delivered smooth muscle cell layers and epithelial layers, sequentially delivered smooth muscle cell layers, gel layers, endothelial or epithelial layers, sequentially delivered pericyte layers and endothelial layers, sequentially delivered pericyte layers and epithelial layers, and/or the like. The cells can be, without limitation, liver cells, cardiac cells, kidney cells, brain cells, gut cells, lung cells, cancer cells, interstitial cells, and/or the like. In various embodiments, the container 130 can be a hermetically scalable container.

In various embodiments, as discussed above, the first reservoir 110 and the second reservoir 120 may be connected to each other by the fluid channel 140 that passes via the container 130 such that the fluid channel 140 and the container 130 are in fluid communication with each other. That is, for example, the second fluid channel section 140b of the fluid channel 140 may be in proximity to, or may make contact with, the container 130 such that fluid communication can be established therebetween to allow the first fluid flowing through the fluid channel 140 (e.g., through the first fluid channel section 140a) to enter the container 130, and/or effluent (e.g., any waste, cellular by-products, etc.) produced by the specimen or cells in the container 130 (referred hereinafter as “cell products”) to diffuse out of the container 130, enter the fluid channel 140, and flow via the third fluid channel section 140c towards the second reservoir 120.

In various embodiments, the second fluid channel section 140b of the fluid channel 140 (e.g., the section that is in proximity or in contact with the container 130) may be or include a portion that is semi-permeable or permeable (e.g., a permeable or semi-permeable membrane) that allows the first fluid flowing therein to diffuse out of the second fluid channel section 140b. Further, the container 130 may be defined by a semi-permeable or permeable material, or may include a portion that is semi-permeable or permeable so as to allow the first fluid diffusing out of the second fluid channel section 140b to diffuse into the container 130 (e.g., and further allow cell products from the container 130 to diffuse out of the container 130 into the fluid channel 140 (e.g., into the second fluid channel section 140b and the third fluid channel section 140c)). For example, the second fluid channel section 140b may have an inner wall 170 that is a semi-permeable or permeable membrane and the container 130 may be defined by, or have, a semi-permeable or permeable membrane wall 180. In such cases, the inner surface 170 may be configured to allow the first fluid in the second fluid channel section 140b to diffuse out of the second fluid channel section 140b, and the semi-permeable or permeable membrane wall 180 may be configured to allow this first fluid to diffuse into the container 130. The semi-permeable or permeable membrane wall 180 may also be configured to allow effluent such as cell products to diffuse out of the container 130 into the fluid channel 140 (e.g., into the second fluid channel section 140b and the third fluid channel section 140c).

In various embodiments, the container 130 and/or the inner wall 170 of the second fluid channel section 140b that is in proximity to or in contact with the container 130 may be made from or include materials such as but not limited to gel, hydrogel (e.g., solid hydrogel), polymerizable hydrogel, collagen methacrylate, silk methacrylate, hyaluronic acid methacrylate, chondroitin sulfate methacrylate, elastin methacrylate, cellulose acrylate, dextran methacrylate, heparin methacrylate, N-isopropyl acrylamide (NIPAAm) methacrylate, Chitosan methacrylate, polyethylene glycol norbornene, polyethylene glycol dithiol, thiolated gelatin, thiolated chitosan, thiolated silk, PEG based peptide conjugates, and/or the like, or any combination thereof. Further, the container 130 and/or the inner wall 170 may be manufactured by applying an additive manufacturing technique to any of the afore-mentioned suitable materials. The additive manufacturing technique may include one or more of a CAL technique, a 3D printing technique such as but not limited to injection molding technique, rapid casting, sacrificial molding, and/or the like. In some instances, the other parts of the fluid channel 140 (e.g., such as the first fluid channel section 140a, the third fluid channel section 140c and the outer wall of the second fluid channel section 140b) may be produced or additively manufactured using a non-permeable material (e.g., plastic), and the inner wall 170 may be additively manufactured (e.g., 3D printed) on the second fluid channel section 140b using any of the afore-mentioned materials (e.g., hydrogels, etc.).

In various embodiments, the container 130 and the fluid channel 140 may be arranged in such a manner that the inner wall 170 is entirely within the interior of the container 130. In such cases, the container 130 may be made from one or more of semi-permeable, permeable, or non-permeable materials, and the inner wall 170 may be made from or include a semi-permeable or permeable material. In such cases, inlet-side fluid flowing in the second fluid channel section 140b may directly diffuse into the interior of the container 130. Further, cell products from then interior of the container 130 may diffuse into the second fluid channel section 140b and flow towards the outlet reservoir 120 via the third fluid channel section 140c.

In some instances, the floor of the container 130 may be non-permeable. In some instances, the floor of the container 130 may be configured to allow optical imaging of the cells or specimen contained within the container from under the container 130. For example, the floor of the container 130 can be translucent or transparent and suitable for imaging with visible light, fluorescence, and/or luminescence. For instance, the floor of the container 130 can be or made from a transparent glass or plastic material, transparent hydrogel, or any other suitable material such as but not limited to polycarbonate, polysulfone, polymethyl methacrylate, polystyrene, cyclic olefin copolymer, polyethylene, polypropylene, glass, quartz, mica, infrared-transparent salts, such as calcium bromide, potassium bromide, any of these materials combined with a thin film of any other material, combination thereof, and/or the like.

In various embodiments, the imaging system 150 may be an optical imaging system that is capable of capturing images of the cells container within the container 130. The imaging system 150 may be positioned under the container 130 to capture images of the cells via the floor of the container 130 that is suitable for imaging with visible light, fluorescence, and/or luminescence.

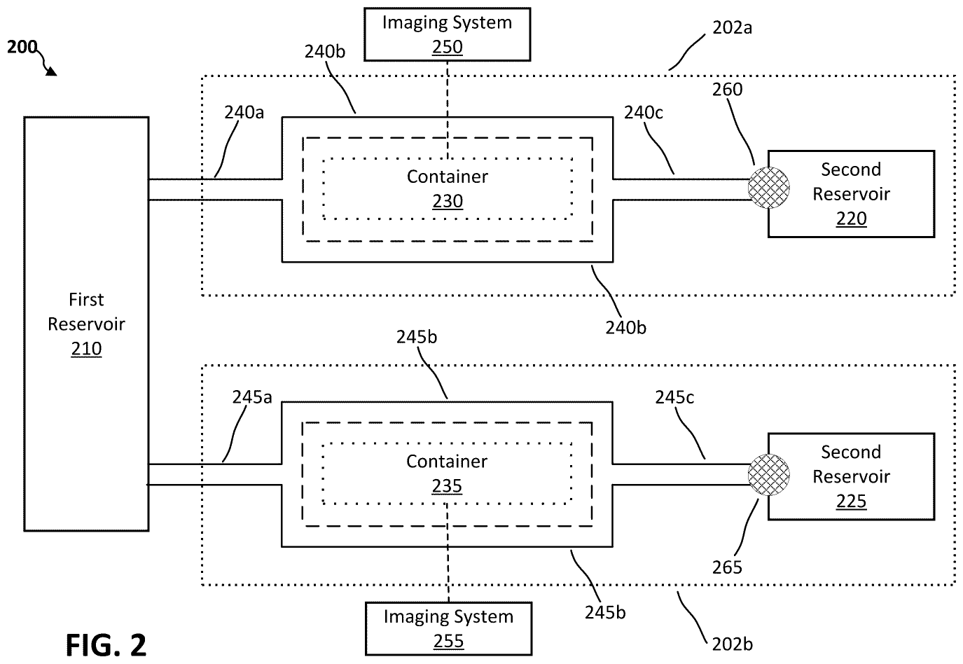

FIG. 2 is a schematic diagram of an system 200 for measuring cell function with a common fluid reservoir in fluid communication with multiple bio-assemblies, according to various embodiments. In various embodiments, the bio-assemblies 202a, 202b may be the same as the bio-assembly 102 of FIG. 1 except that the former do not have respective first reservoirs but rather share a common first reservoir 210. That is, for example, the second reservoirs 220, 225 may be the same as the second reservoir 120, the plugs 260, 265 may be the same as the plug 160, the fluid channels 240, 245 (and the sections 240a, 245a; 240b, 245b; 240c; 245c) may be the same as the fluid channel 140 (and the sections 140a, 140b, 140c, respectively), the containers 230, 235 may be the same as the container 130, and the imaging systems 250, 255 may be the same as the imaging system 150. It is to be understood that, although FIG. 2 shows two bio-assemblies 202a, 202b sharing a single first reservoir 210, it is contemplated that more than two bio-assemblies can share a single first reservoir 210. For example, any number of bio-assemblies in the range from about 2 to about 1,000, from about 4 to about 5,000, from about 8 to about 1,024, from about 16 to about 512, from about 48 to about 256, about 96, including values and subranges therebetween, can share a single first reservoir 210.

In various embodiments, the first reservoir 210 may be configured to receive first fluids that are the same as the first fluids of the first reservoir 110. Further, the second reservoirs 220, 225 may be configured to receive second fluids that are the same as the second fluids of the second reservoir 120. In some instances, the second reservoir 220 and the second reservoir 225 may be configured to receive the same or different second fluids. In either case, the second fluids of the second reservoirs 220, 225 and the first fluid of the first reservoir 210 may be such that entropic gradient force develops therebetween to cause the first fluid to flow out of the first reservoir 210 via the first fluid channel section 240a and the first fluid channel section 245a towards the second reservoir 220 and the second reservoir 225, respectively. For instance, the amount and/or types of the first fluid in the first reservoir 210 and the second fluids in the second reservoirs 220, 225 can be such that entropic gradient force (e.g. osmotic pressure or force) develops between one or both of the second fluids in the second reservoirs 220, 225 and the first fluid in the first reservoir 210 to cause the latter (i.e., the first fluid) to flow out of the first reservoir 210 via the first fluid channel sections 240a, 245a (e.g., in accordance with Bernoulli's principle or equation).

In various embodiments, the entropic gradient force or pressure can be the result of the tendency of the entire system 200 that includes the first reservoir 210, the second reservoirs 220, 225, the containers 230, 235, the fluidic channels 240, 245, etc., to increase its entropy, which can give rise to the entropic gradient force that causes the first fluid in the first reservoir 210 to flow into the first fluid channel sections 240a, 245a. That is, each of the second fluids in the second reservoir 220, which are in fluid communication with the first fluid via fluid connections 240, 245, may be hypertonic with respect to the first fluid, and as such generate the entropic gradient force (e.g., osmotic pressure) on the first fluid to cause the first fluid to flow out of the first reservoir 210, for example, via the first fluid channel section 240a and the container 230 towards the second reservoir 220, and also via the first fluid channel section 245a and the container 235 towards the second reservoir 225.

In various embodiments, the system 200 with the common fluid reservoir 210 for measuring cell function may be used for performing same or similar experiments/measurements on different types, sizes, etc., of specimens. For example, one may wish to study the effects of a first fluid (e.g., therapeutic drugs) on various types of cells (e.g., liver cells, cardiac cells, kidney cells, brain cells, gut cells, etc.). In such cases, each type of cells may be added into different containers 230, 235 of the bio-assemblies 202a, 202b, a first fluid which includes the therapeutic drug (e.g., and in some cases other fluids such as stabilizing agent, bioactive actor, etc.) may be added into the common first reservoir 210, and same or different second fluids that are hypertonic with respect to the first fluid in the first reservoir 210 may be added into the second reservoirs 220 and 225. In such cases, the hypertonicity of the second fluids with respect to the first fluid may cause the first fluid to flow out of the first reservoir 210 via the first fluid channel sections 240a, 245a, and diffuse into the containers 230, 235 as discussed above with reference to FIG. 1. Once diffusion takes place, one may perform measurements on the cells contained in the containers 230, 235 using the imaging systems 250, 255, by retrieving cell samples from the containers 230, 235, and/or by retrieving cell product samples from the second reservoirs 220, 225 after the effluent containing cell products are released from the containers 230, 235 into the fluid channels 240, 245 and flow into the outlet reservoirs 220, 225. In some instances, the “release” of cell products from a container may refer to the cell products diffusing out of the container and entering a fluid channel.

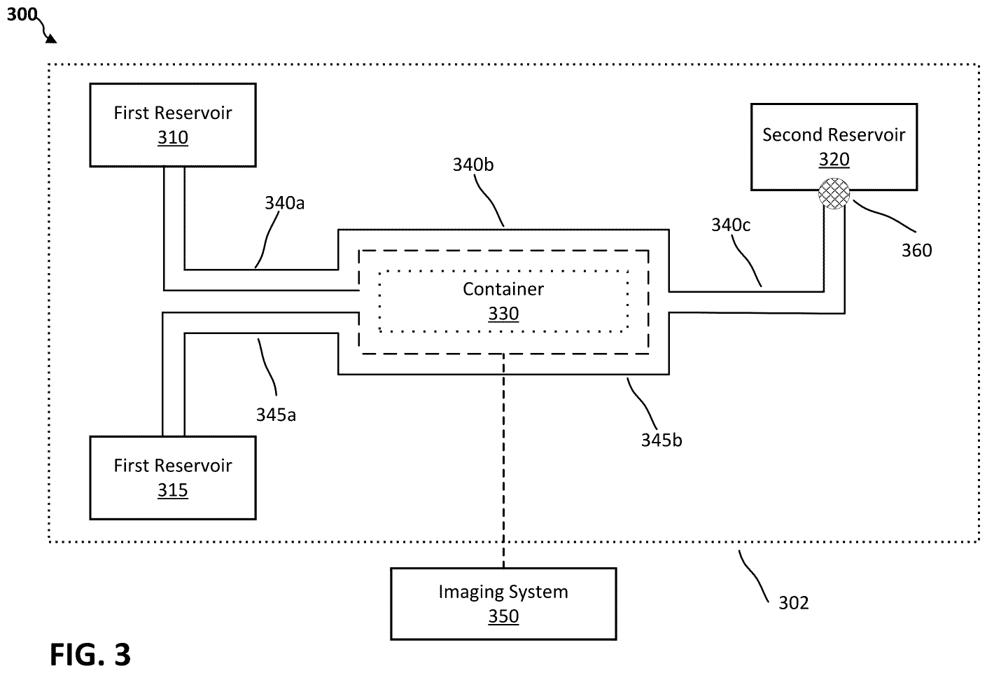

FIG. 3 is a schematic diagram of an system 300 for measuring cell function with multiple fluid reservoirs in fluid communication with a single container of a bio-assembly, according to various embodiments. In various embodiments, the bio-assembly 302 may be the same as the bio-assembly 102 of FIG. 1 except that the former has multiple fluid reservoirs 310, 315, where each is connected to the container 330 via respective first fluid channel sections 340a, 345a. That is, for example, the second reservoir 320 may be the same as the second reservoir 120, the plug 360 may be the same as the plug 160, the first fluid channel sections 340a, 345a may be the same as the first fluid channel section 140a, the second fluid channel section 340b may be the same as the second fluid channel section 140b, the third fluid channel section 340c may be the same as the third fluid channel section 140c, the container 330 may be the same as the container 130, and the imaging system 350 may be the same as the imaging system 150. It is to be understood that, although FIG. 3 shows the bio-assembly 300 having two fluid reservoirs 310, 315 (e.g., with corresponding two first fluid channel sections 340a, 345a), it is contemplated that the bio-assembly 300 can have more than two fluid reservoirs 310, 315 in fluid communication with a single container 330. For example, the system 300 can have any number of first fluid reservoirs ranging from about 2 to about 10, from about 3 to about 8, from about 4 to about 6, about 5, including values and subranges therebetween.

In various embodiments, the second reservoir 320 may be configured to receive second fluid that is the same as the second fluid received by the second reservoir 120. Further, the first reservoirs 310, 315 may be configured to receive first fluids that are the same as the first fluids of the first reservoir 110. In some instances, the first reservoir 310 and the first reservoir 315 may be configured to receive the same or different first fluids. In either case, the first fluids of the inlet reservoirs 310, 315 and the second fluid of the outlet reservoir 320 may be such that entropic gradient force develops therebetween to cause the first fluid to flow out of each of the first reservoirs 310, 315 via the first fluid channels sections 340a, 345a, respectively. For instance, the amount and/or types of the first fluids in the first reservoirs 310, 315 and the second fluid in the second reservoir 320 can be such that entropic gradient force (e.g. osmotic pressure or force) develops between one or both of the first fluids in the first reservoirs 310, 315 and the second fluid in the second reservoir 320 to cause the one or both of the first fluids in the first reservoirs 310, 315 to flow out of their respective first reservoirs 310, 315 via the respective first fluid channel sections 340a, 345a (e.g., in accordance with Bernoulli's principle or equation) towards the second reservoir 320.

In various embodiments, the entropic gradient force or pressure can be the result of the tendency of the entire system 300 that includes the first reservoirs 310, 315, the second reservoir 320, the container 330, the fluidic channels 340, 345a, 345b, to increase its entropy, which can give rise to the entropic gradient force that causes the first fluids in the first reservoirs 310, 325 to flow into the first fluid channel sections 340a, 345a, respectively. That is, the second fluid in the second reservoir 320, which is in fluid communication with the first fluid of first reservoir 310 via fluid channels 340a, 340b, 340c, may be hypertonic with respect to the first fluid of the first reservoir 310, and as such generate entropic gradient force (e.g., osmotic pressure or force) on the first fluid in the first reservoir 310 to cause the first fluid to flow out of the inlet reservoir 310, for example, via the first fluid channel section 340a and the container 330 towards the second reservoir 320. Similarly, the second fluid, which is also in fluid communication with the first fluid of first reservoir 315 via fluid connections 345a, 345b, 340c, may be hypertonic with respect to the first fluid of the first reservoir 310, and as such generate entropic gradient force (e.g., osmotic pressure or force) on the first fluid in the first reservoir 315 to cause the first fluid to flow out of the first reservoir 315, for example, via the first fluid channel section 345a and the container 330 towards the second reservoir 320.

In various embodiments, the system 300 with the multiple first fluid reservoirs 310, 315 may be used for performing experiments/measurements on specimens where the experiments call for the use of multiple first fluids. For example, one may wish to study the effects of a combination of fluids on cells (e.g., liver cells, cardiac cells, kidney cells, brain cells, gut cells, etc.), such as a combination of a therapeutic drug and cell culture media. In such cases, the cells may be added into the container 330, the therapeutic drug may be added into one of the first reservoirs 310, 315 and the cell culture media may be added into the other of the first reservoirs 310, 315, and a second fluid that is hypertonic with respect to the first fluids of the first reservoirs 310, 315 may be added into the second reservoir 320. In such cases, the hypertonicity of the second fluid with respect to the first fluids of the first reservoirs 310, 315 may cause the first fluids to flow out of the first reservoirs 310, 315 via the first fluid channel sections 340a, 345a, respectively, and diffuse into the container 330 as discussed above with reference to FIG. 1. Once diffusion takes place, one may perform measurements on the cells contained in the container 330 using the imaging system 350, by retrieving cell samples from the containers 330, and/or by retrieving cell product samples from the second reservoir 320 after the cell products are released from the container 330 into the fluid channel 340c to flow and enter into the second reservoir 320.

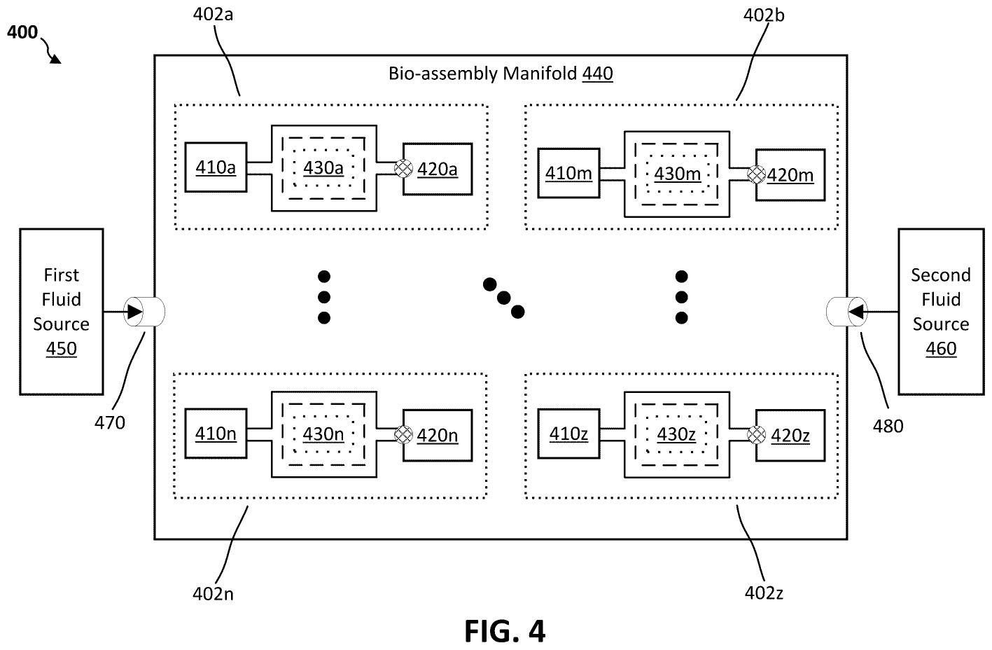

FIG. 4 is a schematic diagram of an example system 400 for measuring cell function, according to various embodiments. In various embodiments, the system 400 may include a bio-assembly manifold 440 of multiple bio-assemblies 402a-402z (collectively labeled “402”), a first fluid source 450, and a second fluid source 460. The bio-assembly manifold 440 can include multiple partitions where each partition is configured to receive a bio-assembly 402. In various embodiments, the bio-assembly 402 may be the same as the bio-assembly 102 of FIG. 1. That is, the first reservoirs 410a-410z may be the same as the first reservoir 110, the containers 430a-430z may be the same as the container 130, and the second reservoirs 420a-420z may be the same as the second reservoir 120. A partition may have a recess (e.g., a void or chamber surrounded by walls, and in some cases a floor) that is sized and shaped to receive the bio-assembly 402. In some cases, a partition may be square-shaped, rectangle-shaped, etc., and the lateral dimensions of the partition (e.g., length, width, depth, etc., of the partition) can be in the range from about 1 mm to about 100 mm, about 5 mm to about 80 mm, about 10 mm to about 60 mm, about 20 mm to about 50 mm, about 30 mm to about 40 mm, including values and subranges therebetween. In some instances, the number of partitions in a bio-assembly manifold 440 can be in the range from about 1 to about 5,000, about 1 to about 1,536, about 2 to about 768, about 4 to about 384, about 6 to about 192, about 8 to about 96, about 12 to about 48, about 16 to about 32, about 24, including values and subranges therebetween.

In various embodiments, the bio-assembly manifold 440 may have a first manifold inlet 470 and a second manifold inlet 480. In some instances, the first manifold inlet 470 can be in fluid communication with an first fluid source 450 to receive first fluids from the first fluid source 450, and the second manifold inlet 480 can be in fluid communication with a second fluid source 460 to receive second fluids from a second fluid source 460. For example, the first fluid source 450 and the second fluid source 460 can be external fluid sources that store the first fluids of the first reservoir 110 and the second fluids of the second reservoir 120, respectively, discussed above with reference to FIG. 1. As a non-limiting example, the first fluid source 450 may store first fluids such as cell culture media, therapeutic drugs, bioactive factors, and/or the like, and the second fluid source 460 may store second fluids that are hypertonic with respect to the first fluids, including but not limited to hypertonic polymer solutions, and/or the like (e.g., and additional solutions such stabilizing reagents, etc.).

As noted above, the first manifold inlet 470 may be in fluidic communication with the first fluid source 450 to receive first fluid stored in the first fluid source 450. The first manifold inlet 470 may also be in fluidic communication with the first reservoirs 410a-410z of the multiple bio-assemblies 402a-402z such the first fluid received by the first manifold inlet 470 from the first fluid source 450 is transported to and deposited into the first reservoirs 410a-410z. For example, the bio-assembly manifold 440 may include a network of first fluid channels that are in fluid communication with the first manifold inlet 470 and the first reservoirs 410a-410z so as to transport the first fluid received by the first manifold inlet 470 from the first fluid source 450 to the first reservoirs 410a-410z and deposit therein.

In some instances, the second manifold inlet 480 may be in fluidic communication with the second fluid source 460 to receive second fluid stored in the second fluid source 460. The second manifold inlet 480 may also be in fluidic communication with the second reservoirs 420a-420z of the multiple bio-assemblies 402a-402z such the second fluid received by the second manifold inlet 480 from the second fluid source 460 is transported to and deposited into the second reservoirs 420a-420z. For example, the bio-assembly manifold 440 may include a network of second fluid channels that are in fluid communication with the second manifold inlet 480 and the second reservoirs 420a-420z so as to transport the second fluid received by the second manifold inlet 480 from the second fluid source 460 to the second reservoirs 420a-420z and deposit therein.

In various embodiments, the bio-assemblies 402a-402z may be removable and replaceable from the bio-assembly manifold 440. For example, once the bio-assemblies 402a-402z are used to conduct experiments or measurements on cells deposited in the containers 430a-430z, a technician may remove the bio-assemblies 402a-4022 from the partitions of the bio-assembly manifold 440 holding the respective bio-assemblies 402a-4022, and replace those with unused bio-assemblies (e.g., in which case the first reservoirs and the second reservoirs of the new bio-assemblies may be filled with first fluid from the first fluid source 450 and second fluid from the second fluid source 460, respectively). In some instances, instead of or in addition to the bio-assemblies 402a-402z being removable and replaceable, one or more of the components of the bio-assemblies 402a-402z may be individually removable and replaceable, for example, the first reservoirs 410a-410z, the containers 430a-430z, and/or the second reservoirs 420a-420z, of the bio-assemblies 402a-402z may be separately removable and replaceable. In some instances, the second reservoirs 420a-420z may be in fluid communication with respective fluid receptacles or a common fluid receptacle such that the fluids in the second reservoirs 420a-420z (e.g., the second fluids as well as first fluids and cell products that have entered into the second reservoirs 420a-420z) may drain into the receptacle(s). In such cases, once the second reservoirs 420a-420z are emptied out into said receptacle(s), the second reservoirs 420a-420z may be filled again with second fluid from the second fluid source 460 for the next experiment or measurement.

### IV. Example Implementation and Application of Cell Function Measurement Systems

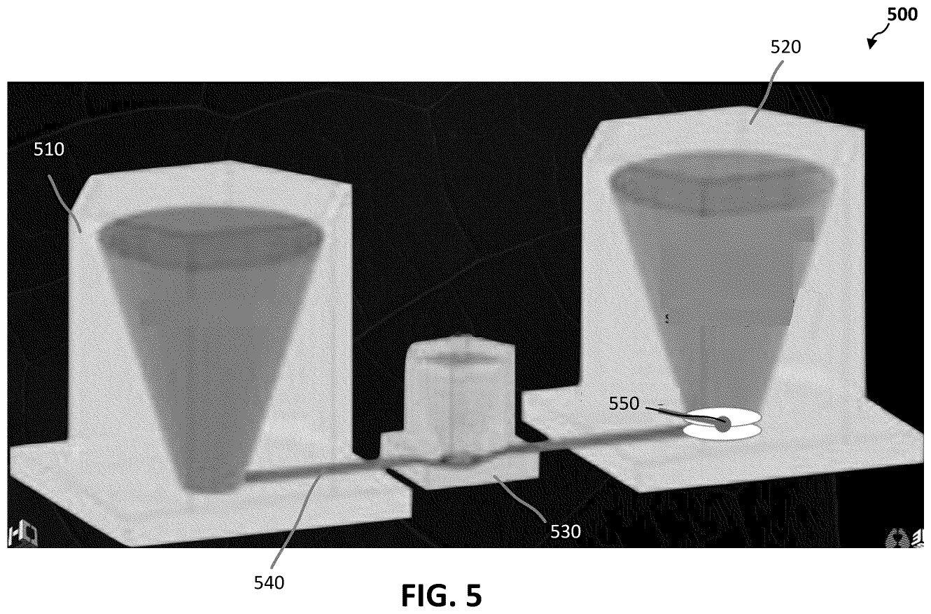

FIG. 5 is an example illustration of a bio-assembly for measuring cell function, according to various embodiments. In various embodiments, the bio-assembly 500 may be the same as the bio-assembly 102 of FIG. 1. That is, the first reservoir 510 may be the same as the first reservoir 110, the container 430 may be the same as the container 130, the plug 550 may be same as the plug 160, and the second reservoir 420 may be the same as the second reservoir 120. In various embodiments, the bio-assembly 500 may be used to conduct biological experiments/measurements where cells are exposed to certain fluidic substances to determine the effect of exposing the cells to the fluidic substances. In general, this may be accomplished by depositing the cells in the container 530, and by adding the fluidic substances (e.g., “first fluid”) and second fluids that are hypertonic relative to the fluidic substances or first fluids into the first reservoir 510 and the second reservoir 520, respectively, such that the hypertonicity of the second fluid with respect to the first fluid causes the first fluid to flow out of the first reservoir 510 via the fluid channel 540 and diffuse into the container in which the cells are disposed.

Examples of such biological investigations for which the bio-assembly 500 can be used include but are not limited to compound screenings for therapeutic drug discovery efforts, etc. For instance, as part of the effort to discover therapeutic drugs to treat various ailments, researchers may wish to screen pharmacologically active compounds by testing said active compounds on target cells looking for desired effects. In other examples, cells (e.g., liver cells, lung cells, kidney cells, etc.) may be exposed to candidate therapeutic drugs to study the effect of the therapeutic drugs on the cells. In such cases, the cells may be added or deposited into the container 530, and the first reservoir 510 may be loaded with a first fluid including the compounds to be screened or the candidate therapeutic drugs. In some instances, the first fluid may also include other cell culture media such as but not limited to nutrients, bioactive factors, etc. Further, the second reservoir 520 may also be loaded with a second fluid that is hypertonic relative to the first fluid. In some instances, the first fluid and the second fluid may be added into their respective reservoirs 510, 520 in either order.

In various embodiments, the first fluid, the second fluid, and/or the bio-assembly 500 may be selected such that equilibrium between the first fluid in the first reservoir 510 and the second fluid in the second reservoir 520 can be reached at least a threshold duration after the transport of the first fluid from the first reservoir 510 towards the second reservoir 520 is initiated. For example, as discussed above, an entropic gradient force such as osmotic force or pressure may develop between the first fluid and the second fluid as a result of the hypertonicity of the second fluid with respect to the first fluid. In such cases, the entropic gradient force may cause the first fluid to flow out of the first reservoir 510 and flow towards the second reservoir 520 via the fluididc channel 540. Equilibrium is reached when there is no net flow of first fluid from the first reservoir 510 towards the second reservoir 520. This occurs because the first fluid in the inlet reservoir 510 as well as the effluent (e.g., cell products) released from the container 530 flow into the second reservoir 520 and render the fluid accumulated in the second reservoir 520 to become less and less hypertonic relative to the first fluid. This results in the entropic gradient force on the first fluid, which is due to the hypertonicity of the second fluid relative to the first fluid, to decrease correspondingly, until the accumulated fluid in the second reservoir 520 no longer becomes hypertonic enough relative to the first fluid to cause the first fluid to flow out of the first reservoir 510 and flow towards the second reservoir 520, resulting in equilibrium being reached between the first fluid in the first reservoir 510 and the accumulated fluid in the second reservoir 520.

For example, the hypertonicity of the second fluid, the size of the fluid channel 540 (e.g., diameter of its cross-section, length, etc.), the volume of the second fluid added into the second reservoir 520, the volume of the first fluid added into the first reservoir 510, and/or the like may be selected such that the afore-mentioned equilibrium is reached no sooner than the threshold duration. With respect to the above examples of testing active compounds or therapeutic drugs, a researcher may wish for an equilibrium to be reached no sooner than 24 hours, i.e., for the threshold duration to be 24 hours. In such cases, the researcher may choose the first fluid and the second fluid such that the hypertonicity of the second fluid relative to the first fluid results in equilibrium between the first fluid and the second fluid being reached no sooner than the threshold duration. Similarly, the bio-assembly 500 may be selected to have a fluid channel 540 that is shaped and sized such that equilibrium is reached no sooner than the threshold duration. As another example, the volumes of the first fluid in the first reservoir 510 and the second fluid in the second reservoir 520 may be selected such that equilibrium is reached no sooner than the threshold duration (e.g., accounting for the changing gravitational potential difference between the fluid in the first reservoir 510 and the fluid in the second reservoir 520 as the first fluid is depleted from the former and enters the latter (e.g., along with cell products released from the container 530)). It is to be noted that the threshold duration can be any desired length of time (as a non-limiting example, the threshold duration can be in the range from about one minute to about one week).

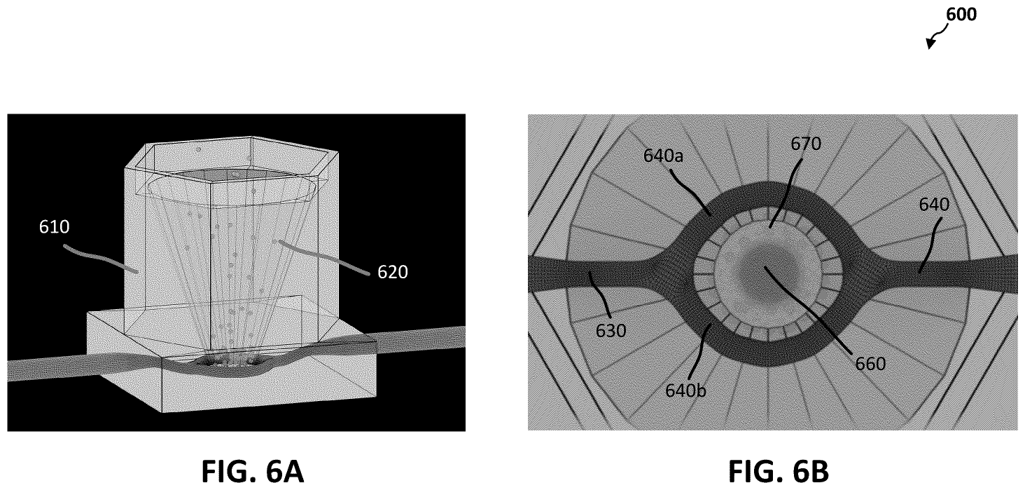

As noted above, the first fluid in the first reservoir 510 flows towards the second reservoir 520 via the fluid channel 540 under an entropic gradient force (e.g., osmotic pressure or force) that is created due to the hypertonicity of the second fluid in the outlet reservoir 520 relative to the first fluid. The fluid channel 540 is in fluid communication with the container 530 as discussed above with reference to FIG. 1, and as such, the first fluid diffuses into the interior of the container 530 as it flows via the fluid channel 540. FIGS. 6A-6B show example illustrations of a container 610 holding cells 620 as fluid from a first reservoir diffuses into the container 610. In various embodiments, cells 620 may be deposited into a container 610 of a bio-assembly (e.g., such as the bio-assembly 500). In some instances, the container 610 can be hermetically scalable, and as such, may be sealed after all the cells 620 are added to avoid contamination.

In various embodiments, the first fluid that is flowing from the first reservoir towards the second reservoir via the first fluid channel section 630 may arrive at the second fluid channel sections 640a, 640b, which may be in proximity to, in contact with, or located within, the container 610 such that the first fluid diffuses into the interior of the container 610 when flowing via the second fluid channel sections 640a, 640b (e.g., as discussed above with reference to container 130 of FIG. 1). For example, the second fluid channel sections 640a, 640b may be semi-permeable or permeable and may allow the first fluid to diffuse out of the second fluid channel sections 640a, 640b and enter the interior of the container 610. In some instances, the second fluid channel sections 640a, 640b may not be positioned within the interior of the container 610 (e.g., may be in contact with or in proximity to the container 610), and in such cases, the walls of the container 610 may be semi-permeable or permeable, and as such may allow the first fluid that has diffused out of the second fluid connection sections 640a, 640b to diffuse into the interior of the container 610.

FIG. 6B shows an example snapshot of a cross-sectional view of the floor of the container 610 after some time has passed since the first fluid started diffusing into the interior of the container 610. For example, the length of this time period can be no shorter than the threshold duration for equilibrium to be reached between the first fluid in the first reservoir and the fluids accumulated in the second reservoir after the flow of the first fluid is initiated. After the diffusion of the first fluid into the interior of the container 610, the cells 620 contained therein may respond to their exposure to first fluid (e.g., and constituents of the first fluid such as cell culture media, therapeutic drugs, bioactive factors or compounds, etc.). For example, the cells 620 may produce cell products such as but not limited to markers 660 of cell apoptosis, exosomes, and/or the like. In other example, the remaining alive cells 670 may display effects of the first fluid (e.g., cell behaviors or effects caused by the first fluid including a therapeutic drug).

In various embodiments, measurements may be performed on the cells or cell products (e.g., markers 660 of cell apoptosis, exosomes, etc., remaining alive cells 670, etc.) by retrieving samples thereof from the container 610 and analyzing the samples. Further, the cells or cell products in the container 610 may be imaged using an imaging system positioned under the container 610 (e.g., provided the floor of the container 610 is transparent to the optical signals from the imaging system) and the captured images may be analyzed. The analysis of the samples and/or the images may be done as part of the biological investigation for which the bio-assembly is used, e.g., compound screenings for therapeutic drug discovery efforts, etc.

In various embodiments, samples may also be retrieved from the second reservoir of the bio-assembly after effluent is released or discharged from the container 610 into the fluidic channel 640 that transports the effluent (e.g., along with the first fluid from the first reservoir) to the second reservoir. The effluent may cell byproducts produced by the cells in the container 610, markers 660 of cell apoptosis, exosomes, etc., remaining alive cells 670, or combination thereof, and/or the like. That is, for example, the cell products produced by the cells in the container 610 may diffuse out of the container 610 and enter into the fluid channel 640 to flow into the outlet reservoir (e.g., along with first fluid from the first reservoir) of the bio-assembly. As discussed above with reference to FIG. 1, the plug positioned between the fluid connection 640 and the second reservoir can be a semi-permeable membrane configured to allow the cell products and the first fluid enter into the second reservoir while preventing fluids (e.g., hypertonic fluids with high molecular weight) in the second reservoir from flowing out of the second reservoir via the fluid channel 640. In such cases, sample volumes from the second reservoir (e.g., which may include the cell byproducts and first fluids that diffused into the second reservoir as well as the hypertonic solution and other constituents of the second fluid) may be retrieved and measured/analyzed (e.g., as part of the biological investigation for which the bio-assembly is used, e.g., compound screenings for therapeutic drug discovery efforts, etc.).

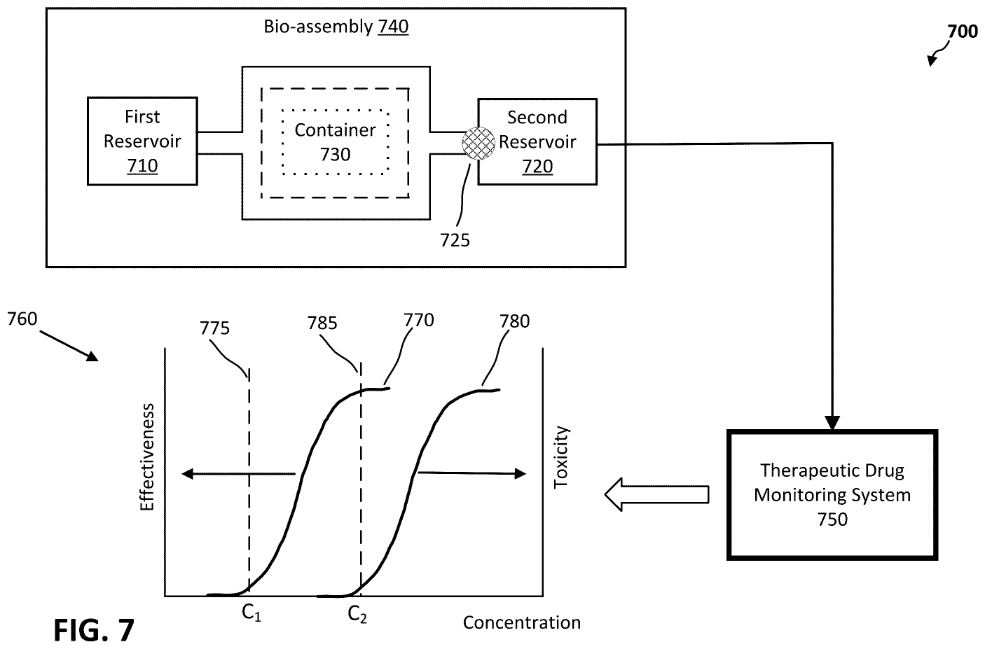

FIG. 7 is a schematic workflow 700 for determining the therapeutic window for a drug, according to various embodiments. In various embodiments, the therapeutic window of a drug refers to the range of drug concentrations in which the drug is effective without being toxic. In general, most drugs can be ineffective at low concentration while having adverse effects at high concentrations. The therapeutic window of a drug is then the range of drug concentration between the minimum effective concertation of the drug (e.g., the minimum concentration of the drug at which the drug has therapeutic effect or elicits a therapeutic response) and the minimum toxicity concentration of the drug (e.g., the minimum concentration of the drug at which the drug causes adverse effect or toxicity).

In various embodiments, the bio-assembly 740 including the first reservoir 710, the second reservoir 720, the plug 725, and the container 730 may be used to determine the therapeutic window of drugs. In various embodiments, the bio-assembly 740 may be the same as the bio-assembly 102 of FIG. 1. That is, the first reservoir 710 may be the same as the first reservoir 110, the container 730 may be the same as the container 130, the second reservoir 720 may be the same as the second reservoir 120, and the plug 725 may be the same as the plug 160. The plug 725 may be configured to prevent the second fluid from flowing out of the second reservoir 720 while allowing fluids to enter the second reservoir 720. For example, the plug 725 can be a semi-permeable membrane (e.g., solid hydrogel).

In some instances, a first fluid including the drug, for which the therapeutic window is being determined using the bio-assembly 740 of FIG. 7, may be added into the first reservoir 710. Additional fluids such as but not limited to cell culture media (e.g., nutrients, etc.) may also be added into the first reservoir 710. Further, a second fluid that is hypertonic relative to the first fluid of the first reservoir 710 may be added into the second reservoir 720. For determining the therapeutic window of a drug, blood and/or plasma samples are added into the container 730.

In various embodiments, as discussed in more details above (e.g., with reference to FIGS. 5 and 6A-6B), the first fluid may flow out of the first reservoir 710 under the entropic gradient force (e.g., osmotic pressure or force) caused by the hypertonicity of the second fluid, in the second reservoir 720, with respect to the first fluid. This flow may continue until equilibrium is established between the first fluid in the first reservoir 710 and the fluid that is accumulated in the second reservoir 720. The duration between the start of the flow of the first fluid out of the first reservoir 710 and the establishment of the equilibrium between the first fluid and the second fluid can depend on the hypertonicity of the second fluid relative to the first fluid, the size (e.g., cross-sectional area) of the fluid channel connecting the first reservoir 710 to the second reservoir 720, the volumes of the first fluid and the second fluid, and/or the like. Effluent released by the container 730 may include cell products produced by the cells/tissues disposed in the container 730 after the cells are exposed to the first fluid that diffuses into the container 730. That is, the first fluid from the first reservoir 710 may diffuse into the container 730 and the cells therein may be exposed to the components of the first fluid such as therapeutic drugs, nutrients, etc. Cell byproducts such as but not limited to markers of cell apoptosis, exosomes, etc., may then be released out of the container 730 (e.g., diffuse out of the container 730), flow via the fluid channel and enter into the second reservoir 720 (e.g., diffusing through the semi-permeable plug 725).

In various embodiments, samples may be retrieved from the container 730 (e.g., before the cell products diffuse out of the container 730) and/or the second reservoir 720 (e.g., after the cell products enter the second reservoir 720) to determine the therapeutic window of the drug. For example, a therapeutic drug monitoring system 750 may be used to analyze the samples to determine the therapeutic window of the drug. Non-limiting examples of such therapeutic drug monitoring systems 750 include systems implementing one or more analytical tools such as but not limited to liquid chromatography-mass spectrometry, gas chromatography-mass spectrometry, high-performance liquid chromatography, and/or the like that are capable of analyzing the samples and determining the effectiveness and/or toxicity of the therapeutics drug. For example, the therapeutic drug monitoring system 750 may determine from the sample the minimum effective concentration of the therapeutic drug at which the drug has an effect or triggers a therapeutic response from the cells/tissues and the minimum toxic concentration of the therapeutic drug at which the drug starts to have an adverse effect or toxicity on the cells/tissues. The therapeutic window is then the window or range of drug concentration between the minimum effective concentration and the minimum toxic concentration. For example, FIG. 7 shows an example schematic plot 760 including the effectiveness curve 770 and the toxicity curve 780 of a therapeutic drug as a function of concentration. The concentration C₁ 775 and the concentration C₂ 785 correspond to the minimum effective concentration and the minimum toxic concentration, respectively, of the drug, and the therapeutic window of the drug may then be determined as the drug concentration range from about concentration C₁ 775 to about concentration C₂ 785.

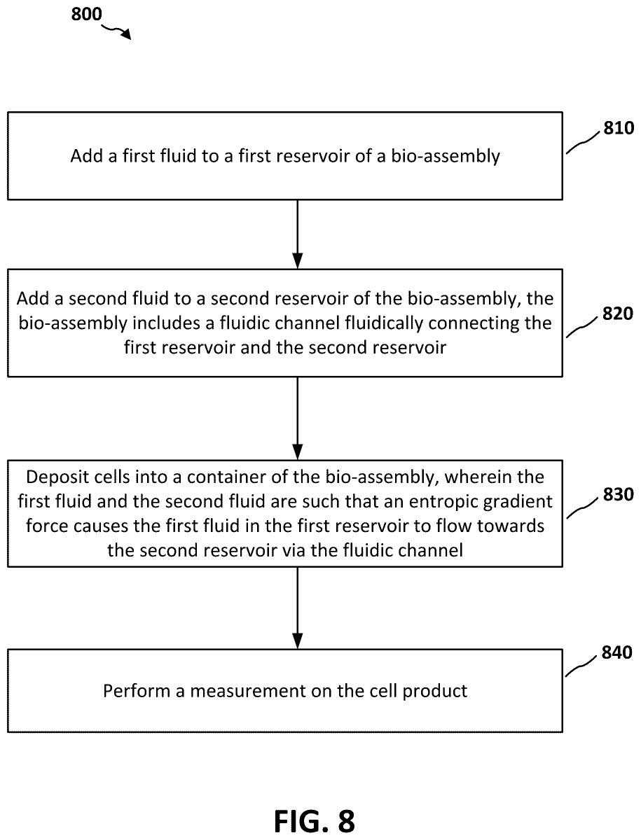

FIG. 8 is a flowchart illustrating a method 800 for measuring cell function, according to various embodiments. Aspects of the method 800 can be executed by the cell function measurement systems 100, 200, 300, 400, 500, the bio-assemblies 600, 700, or other suitable means for performing the steps. As illustrated, the method 800 includes a number of enumerated steps, but aspects of the method 800 may include additional steps before, after, and in between the enumerated steps. In some embodiments, one or more of the enumerated steps may be omitted or performed in a different order.

At block 810, a first fluid is added to a first reservoir of a bio-assembly.

At block 820, a second fluid is added into a second reservoir of the bio-assembly. In various embodiments, the bio-assembly includes a fluidic channel connecting the first reservoir and the second reservoir, and a plug positioned between the second reservoir and the fluidic channel, the plug configured to prevent the outlet-side fluid from flowing out of the outlet reservoir via the fluidic connection. In various embodiments, the plug can be a semi-permeable membrane made from a solid hydrogel material. In various embodiments, the first fluid and the second fluid are such that an entropic gradient force causes the first fluid in the first reservoir to flow towards the second reservoir via the fluidic channel.

At block 830, cells are deposited into a container of the bio-assembly. In various embodiments, the container is in fluidic communication with the fluidic channel to (i) intake a portion of the first fluid flowing in the fluidic channel from the first reservoir towards the second reservoir; and (ii) release a cell product produced by the cells into the first fluid flowing in the fluidic channel towards the second reservoir. In various embodiments, the cells can be liver cells, kidney cells, and/or the like. In various embodiments, the cell product can include markers of cell apoptosis, exosomes, and/or the like.

At block 840, a measurement is performed on the cell product produced by the cells and released into the first fluid flowing in the fluid channel towards the second reservoir. In various embodiments, the measurement of the cell product includes imaging, using an imaging system positioned in proximity to the container, the cell product within the container. In some instances, the imaging system can be a fluorescence imaging system, and/or the like. In various embodiments, the measurement of the cell product includes retrieving a first sample of the cell product from the container prior to the release of the cell product into the inlet-side fluid flowing towards the second reservoir. In various embodiments, the measurement of the cell product includes retrieving a second sample of the cell product from the second reservoir after the cell product released into the first fluid flowing in the fluidic channel towards the second reservoir enter the second reservoir.

In various embodiments, the inlet-side fluid includes cell culture media, a therapeutic drug, or a bioactive factor. For example, the therapeutic drug can include a chemotherapeutic drug, an antibiotic, and/or the like. As another example, bioactive factor can include a growth factor, an intracellular signaling molecule, or a signaling mimetic. In various embodiments, the cell culture media can include nutrient, a glucose solution, a serum, an amino acid, an inorganic salt, or a vitamin. In various embodiments, the outlet-side fluid includes a hypertonic polymer solution, a stabilizing reagent, or a combination thereof. For example, the hypertonic polymer solution can include a polyethylenimine (PEI) solution, or a combination thereof. As another example, the stabilizing reagent can include a phosphate or an amorphous calcium carbonate.

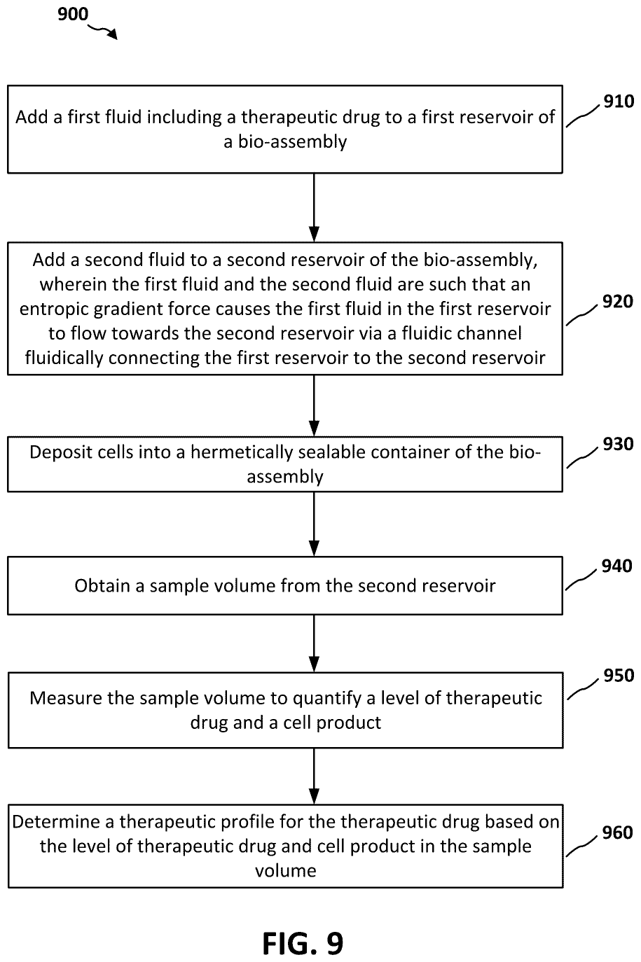

FIG. 9 is a flowchart illustrating a method 900 for determining an optimal therapeutic window for a therapeutic drug, according to various embodiments. Aspects of the method 900 can be executed by the cell function measurement systems 100, 200, 300, 400, 500, the bio-assemblies 600, 700, or other suitable means for performing the steps. As illustrated, the method 900 includes a number of enumerated steps, but aspects of the method 900 may include additional steps before, after, and in between the enumerated steps. In some embodiments, one or more of the enumerated steps may be omitted or performed in a different order.

At block 910, a first fluid including a therapeutic drug is added into an inlet reservoir of a bio-assembly.

At block 920, a second fluid is added into the outlet reservoir of the bio-assembly. In various embodiments, the first fluid and the second fluid are such that an entropic gradient force causes the first fluid in the first reservoir to flow towards the second reservoir via a fluidic channel fluidically connecting the first reservoir to the second reservoir. For example, the second fluid can be hypertonic with respect to the first fluid such that the hypertonicity of the outlet-side fluid creates the entropic gradient force on the inlet-side fluid that causes the first fluid to flow out of the first reservoir and towards the second reservoir. In various embodiments, the hypertonic outlet-side fluid includes a hypertonic polymer solution, a stabilizing reagent, or a combination thereof. For example, the hypertonic polymer solution can include a polyethylenimine (PEI) solution, or a combination thereof. As another example, the stabilizing reagent can include a phosphate or an amorphous calcium carbonate.

At block 930, cells are deposited into a hermetically scalable container of the bio-assembly. In various embodiments, the hermetically scalable container is in fluidic communication with the fluidic channel to intake a portion of the first fluid including the therapeutic drug flowing in the fluidic channel from the first reservoir. In various embodiments, the fluidic communication between the container and the fluidic channel allows the container to release cell products produced by the cells into the first fluid flowing in the fluidic channel towards the second reservoir.

At block 940, a sample volume is obtained from the second reservoir. In various embodiments, obtaining the sample volume from the second reservoir includes retrieving from the second reservoir cell products, produced by the cells in the container, after the cell products are released into the first fluid flowing in the fluidic channel towards the second reservoir and enter the second reservoir.

At block 950, the sample volume is measured to quantify a level of therapeutic drug and a cell product. In various embodiments, the level of the therapeutic drug and the cell product can include a concentration of the therapeutic drug in the container and/or in the second reservoir after the released cell products arrive in the second reservoir, the amount of the cell product in the second reservoir, etc.

At block 960, a therapeutic profile for the therapeutic drug is determined based on the level of therapeutic drug and cell product in the sample volume. In various embodiments, the therapeutic profile of the therapeutic drug can be or include the therapeutic window of the drug. In various embodiments, the therapeutic profile or the therapeutic window of the therapeutic drug is determined using the cell product and therapeutic drug concentration by systems implementing one or more analytical tools such as but not limited to liquid chromatography-mass spectrometry, gas chromatography-mass spectrometry, high-performance liquid chromatography, and/or the like that are capable of analyzing the sample volume and determining the minimum effective concentration and the minimum toxic concentration of the therapeutic drug, from which the therapeutic window may be determined.

While the present teachings are described in conjunction with various embodiments, it is not intended that the present teachings be limited to such various embodiments. On the contrary, the present teachings encompass various alternatives, modifications, and equivalents, as will be appreciated by those of skill in the art.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

### V. Additional Recited Embodiments

Embodiment 1: A bio-assembly, comprising: a container defined within a semi-permeable material; a first reservoir configured to receive a first fluid; a second reservoir configured to receive a second fluid; a fluidic channel fluidically connecting the first reservoir and the second reservoir, wherein the first fluid and the second fluid are such that an entropic gradient force causes the first fluid in the first reservoir to flow towards the second reservoir via the fluidic channel; and a plug positioned between the second reservoir and the fluidic channel, the plug configured to prevent the second fluid from flowing out of the second reservoir via the fluidic connection, wherein: the container is in fluidic communication with the fluid channel and is configured to (i) intake a portion of the first fluid flowing in the fluidic channel from the first reservoir towards the second reservoir; and (ii) release effluent from the container into the first fluid flowing in the fluidic channel towards the second reservoir.
Embodiment 2: The bio-assembly of embodiment 1, wherein the plug is configured to prevent solutes in the second fluid having molecular weight greater than a threshold molecular weight from diffusing therethrough.
Embodiment 3: The bio-assembly of embodiment 2, wherein the threshold molecular weight is about 5 kDa.
Embodiment 4: The bio-assembly of any of the preceding embodiments, wherein the plug is a semi-permeable membrane.
Embodiment 5: The bio-assembly of any of the preceding embodiments, wherein the plug is made from a hydrogel material, or a combination thereof.
Embodiment 6: The bio-assembly of any of the preceding embodiments, wherein the semi-permeable material of the container is made from a hydrogel material.
Embodiment 7: The bio-assembly of any of the preceding embodiments, wherein the inlet reservoir, the outlet reservoir, and/or the fluidic connection are made from a plastic material.
Embodiment 8: The bio-assembly of any of the preceding embodiments, wherein the first reservoir, the second reservoir, and/or the fluidic channel are components of a monolithic manifold.
Embodiment 9: The bio-assembly of any of the preceding embodiments, wherein the first reservoir, the second reservoir, the container, and/or the fluidic channel are 3D-printed or molded using a 3D printing technique.
Embodiment 10: The bio-assembly of embodiment 9, wherein the 3D printing technique includes an injection molding technique, a rapid casting or sacrificial molding technique, or a computed axial lithography (CAL) technique.
Embodiment 11: The bio-assembly of any of the preceding embodiments, wherein the container is a generally conic-shaped container with an opening at a wider of the two bases of the generally conic-shaped container configured for receiving cells into the container.
Embodiment 12: The bio-assembly of any of the preceding embodiments, wherein the entropic gradient force is due at least in part to a hypertonicity of the outlet-side fluid with respect to the inlet-side fluid.
Embodiment 13: The bio-assembly of any of the preceding embodiments, wherein equilibrium between the first fluid in the first reservoir and the second fluid in the second reservoir is reached at least a threshold duration after the flow of the first fluid from the first reservoir towards the second reservoir is initiated due to the entropic gradient force.
Embodiment 14: The bio-assembly of embodiment 13, wherein the threshold duration depends on a size of the fluidic channel and/or a hypertonicity of the second fluid in the second reservoir.
Embodiment 15: The bio-assembly of embodiment 13 or 14, wherein the threshold duration is about twenty four hours.
Embodiment 16: The bio-assembly of any of the preceding embodiments, wherein the effluent includes cell by-products of cells disposed within the container.
Embodiment 17: The bio-assembly of embodiment 16, wherein the cells include liver cells.
Embodiment 18: The bio-assembly of any of the preceding embodiments, wherein the first fluid includes cell culture media, a therapeutic drug, or a bioactive factor.
Embodiment 19: The bio-assembly of embodiment 18, wherein the cell culture media include a glucose solution, a serum, an amino acid, an inorganic salt, or a vitamin.
Embodiment 20: The bio-assembly of embodiment 18 or 19, wherein the therapeutic drug includes a chemotherapeutic drug or an antibiotic.
Embodiment 21: The bio-assembly of any of embodiments 18-20, wherein the bioactive factor includes a growth factor, an intracellular signaling molecule, or a signaling mimetic.
Embodiment 22: The bio-assembly of any of the preceding embodiments, wherein the second fluid includes a hypertonic polymer solution, a stabilizing reagent, or a combination thereof.
Embodiment 23: The bio-assembly of embodiment 22, wherein the hypertonic polymer solution includes a polyethylenimine (PEI) solution.
Embodiment 24: The bio-assembly of any of the preceding embodiments, wherein the first fluid includes a third fluid and a fourth fluid, and the first reservoir includes a third reservoir configured to receive the third fluid and a fourth reservoir configured to receive the fourth fluid.
Embodiment 25: The bio-assembly of embodiment 24, wherein the third fluid includes one of a cell culture media, a therapeutic drug, or a bioactive factor, and the fourth fluid includes another one of the cell culture media, the therapeutic drug, or the bioactive factor.
Embodiment 26: The bio-assembly of any of embodiments 1-23, wherein the container includes a first container and a second container, the second reservoir includes a third reservoir and a fourth reservoir, the fluidic channel includes a first channel between the first container and the third reservoir and a second channel between the second container and the fourth reservoir.
Embodiment 27: A method for measuring cell function, comprising: adding a first fluid to an inlet reservoir of a bio-assembly; adding a second fluid to an outlet reservoir of the bio-assembly; and depositing cells into a container of the bio-assembly, wherein: the bio-assembly includes a fluidic channel fluidically connecting the first reservoir and the second reservoir; the first fluid and the second fluid are such that an entropic gradient force causes the first fluid in the first reservoir to flow towards the second reservoir via the fluidic channel; the bio-assembly further includes a plug positioned between the second reservoir and the fluidic channel, the plug configured to prevent the second fluid from flowing out of the second reservoir via the fluidic channel; and the container is in fluidic communication with the fluidic channel to (i) intake a portion of the first fluid flowing in the fluidic channel from the first reservoir towards the second reservoir; and (ii) release a cell product produced by the cells into the first fluid flowing in the fluidic channel towards the second reservoir; and performing a measurement on the cell product.
Embodiment 28: The method of embodiment 27, wherein the performing includes imaging, using an imaging system positioned in proximity to the container, the cell product within the container.
Embodiment 29: The method of embodiment 28, wherein the imaging system includes a fluorescence imaging system.
Embodiment 30: The method of any of embodiments 27-29, wherein the performing includes retrieving a first sample of the cell product from the container prior to the release of the cell product into the first fluid flowing in the fluidic channel towards the outlet reservoir.
Embodiment 31: The method of any of embodiments 27-30, wherein the performing includes retrieving a second sample of the cell product from the second reservoir after the cell product released into the first fluid flowing in the fluidic channel towards the outlet reservoir enter the outlet reservoir.
Embodiment 32: The method of any of embodiments 27-31, wherein the cell product includes markers of cell apoptosis, or exosomes.
Embodiment 33: The method of any of embodiments 27-32, wherein the plug is a semi-permeable membrane made from a solid hydrogel material.
Embodiment 34: The method of any of embodiments 27-33, wherein the entropic gradient force is due at least in part to a hypertonicity of the second fluid with respect to the first fluid.
Embodiment 35: The method of any of embodiments 27-34, wherein the cells include liver cells.
Embodiment 36: The method of any of embodiments 27-35, wherein the first fluid includes cell culture media, a therapeutic drug, or a bioactive factor.
Embodiment 37: The method of any of embodiments 27-36, wherein the cell culture media include a glucose solution, a serum, an amino acid, an inorganic salt, or a vitamin.
Embodiment 38: The method of embodiment 36 or 37, wherein the therapeutic drug includes a chemotherapeutic drug or an antibiotic.
Embodiment 39: The method of any of embodiments 27-38, wherein the bioactive factor includes a growth factor, an intracellular signaling molecule, or a signaling mimetic.
Embodiment 40: The method of any of embodiments 27-39, wherein the outlet-side fluid includes a hypertonic polymer solution, a stabilizing reagent, or a combination thereof.
Embodiment 41: The method of embodiment 40, wherein the hypertonic polymer solution includes a polyethylenimine (PEI) solution.
Embodiment 42: The method of embodiment 40 or 41, wherein the stabilizing reagent includes a phosphate or an amorphous calcium carbonate.
Embodiment 43: A method for determining an optimal therapeutic window for a therapeutic drug, comprising: adding a first fluid including a therapeutic drug to a first reservoir of a bio-assembly; adding a second fluid to a second reservoir of the bio-assembly, wherein the first fluid and the second fluid are such that an entropic gradient force causes the first fluid in the first reservoir to flow towards the second reservoir via a fluidic channel fluidically connecting the first reservoir to the second reservoir; depositing cells into a hermetically scalable container of the bio-assembly; obtaining a sample volume from the second reservoir; measuring the sample volume to quantify a level of therapeutic drug and a cell product; and determining a therapeutic profile for the therapeutic drug based on the level of therapeutic drug and cell product in the sample volume.
Embodiment 44: The method of embodiment 43, wherein: the bio-assembly includes a plug positioned between the second reservoir and the fluidic channel, the plug configured to prevent the second fluid from flowing out of the second reservoir via the fluidic channel; and the container is in fluidic communication with the fluidic channel to intake a portion of the first fluid including the therapeutic drug flowing in the fluidic channel from the first reservoir to the second reservoir.
Embodiment 45: The method of embodiment 43 or 44, wherein the fluidic communication between the hermetically sealable container and the fluidic channel is configured to allow the hermetically sealable container to release cell products produced by the cells in the container into the first fluid flowing in the fluidic channel towards the second reservoir.
Embodiment 46: The method of any of embodiments 43-46, wherein the obtaining the sample volume from the second reservoir includes retrieving from the second reservoir cell products, produced by the cells in the hermetically sealable container, after the cell products are released into the second fluid flowing in the fluidic channel towards the outlet reservoir and enter the second reservoir.
Embodiment 47: The method of any of embodiments 43-46, wherein the therapeutic drug includes a chemotherapeutic drug or an antibiotic.
Embodiment 48: The method of any of embodiments 43-47, wherein the hypertonic outlet-side fluid includes a hypertonic polymer solution, a stabilizing reagent, or a combination thereof.
Embodiment 49: The method of embodiment 48, wherein the hypertonic polymer solution includes a polyethylenimine (PEI) solution.
Embodiment 50: The method of embodiment 48 or 49, wherein the stabilizing reagent includes a phosphate or an amorphous calcium carbonate.
Embodiment 51: The method of any of embodiments 43-50, wherein the plug includes a semi-permeable membrane configured to prevent solutes of the second fluid with molecular weight exceeding a threshold molecular weight from flowing out of the second reservoir via the fluidic channel.
Embodiment 52: A system for measuring cell function, comprising: a manifold including a plurality of bio-assemblies, each bio-assembly comprising: a container; a first reservoir configured to receive a first fluid; a second reservoir configured to receive a second fluid; a fluidic channel fluidically connecting the first reservoir to the second reservoir via the container; and a plug positioned between the second reservoir and the fluidic channel, and configured to prevent the second fluid from flowing out of the second reservoir via the fluidic channel, wherein: the first fluid and the second fluid are such that an entropic gradient force causes the first fluid in the first reservoir to flow towards the second reservoir via the fluidic channel; a first fluid source in fluidic communication with the first reservoir to provide the first reservoir with the first fluid; and a second fluid source in fluidic communication with the second reservoir to provide the second reservoir with the second fluid.
Embodiment 53: The system of embodiment 52, wherein each bio-assembly is individually removable from the manifold.
Embodiment 54: The system of embodiment 52 or 53, wherein the second reservoir of each bio-assembly is removable from the bio-assembly and replaceable.
Embodiment 55: The system of any of embodiments 52-54, wherein the plurality of bio-assemblies include 2, 12, 24, 48, or 96 bio-assemblies.
Embodiment 56: The system of any of embodiments 52-55, wherein the container is in fluidic communication with the fluid channel to release cell products produced by cells located therein into the first fluid flowing in the fluidic channel towards the second reservoir.
Embodiment 57: The system of any of embodiments 52-56, wherein the plug is a semi-permeable membrane made from a hydrogel material.
Embodiment 58: A kit, comprising: a container defined within a semi-permeable material; a first reservoir containing a first fluid; a second reservoir containing a second fluid; a fluidic channel fluidically connecting the first reservoir and the second reservoir, wherein the first fluid and the second fluid are such that an entropic gradient force causes the first fluid in the first reservoir to flow towards the second reservoir via the fluidic channel; and a plug positioned between the second reservoir and the fluidic channel, the plug configured to prevent the second fluid from flowing out of the second reservoir via the fluidic channel, wherein: the container is in fluidic communication with the fluid channel and configured to intake a portion of the first fluid flowing in the fluidic channel from the first reservoir and release cell products produced by cells housed in the container into the first fluid flowing in the fluidic channel towards the second reservoir; and equilibrium between the first fluid in the first reservoir and the second fluid in the second reservoir is reached at least a threshold duration after the flow of the first fluid from the first reservoir towards the second reservoir is initiated due to the entropic gradient force.
Embodiment 59: The kit of embodiment 58, wherein the threshold duration depends on a size of the fluidic channel and/or a hypertonicity of the second fluid.
Embodiment 60: The kit of embodiment 58 or 59, wherein the threshold duration is about twenty four hours.

What is claimed is:

1. A bio-assembly, comprising:
a container defined within a semi-permeable material;
a first reservoir configured to receive a first fluid;
a second reservoir configured to receive a second fluid;
a fluidic channel fluidically connecting the first reservoir and the second reservoir, wherein the first fluid and the second fluid are such that an entropic gradient force causes the first fluid in the first reservoir to flow towards the second reservoir via the fluidic channel; and
a plug positioned between the second reservoir and the fluidic channel, the plug configured to prevent the second fluid from flowing out of the second reservoir via the fluidic connection, wherein:
the container is in fluidic communication with the fluid channel and is configured to (i) intake a portion of the first fluid flowing in the fluidic channel from the first reservoir towards the second reservoir; and (ii) release effluent from the container into the first fluid flowing in the fluidic channel towards the second reservoir.

2. The bio-assembly of claim 1, wherein the plug is configured to prevent solutes in the second fluid having molecular weight greater than a threshold molecular weight from diffusing therethrough.

3. The bio-assembly of claim 2, wherein the threshold molecular weight is about 5 kDa.

4. The bio-assembly of claim 1, wherein the plug is a semi-permeable membrane made from a hydrogel material.

5. The bio-assembly of claim 1, wherein the inlet reservoir, the outlet reservoir, and/or the fluidic connection are made from a plastic material.

6. The bio-assembly of claim 1, wherein the first reservoir, the second reservoir, and/or the fluidic channel are components of a monolithic manifold.

7. The bio-assembly of claim 1, wherein the first reservoir, the second reservoir, the container, and/or the fluidic channel are 3D-printed or molded using a 3D printing technique.

8. The bio-assembly of claim 7, wherein the 3D printing technique includes an injection molding technique, a rapid casting or sacrificial molding technique, or a computed axial lithography (CAL) technique.

9. The bio-assembly of claim 1, wherein the container is a generally conic-shaped container with an opening at a wider of the two bases of the generally conic-shaped container configured for receiving cells into the container.

10. The bio-assembly of claim 1, wherein the entropic gradient force is due at least in part to a hypertonicity of the outlet-side fluid with respect to the inlet-side fluid.

11. The bio-assembly of claim 1, wherein equilibrium between the first fluid in the first reservoir and the second fluid in the second reservoir is reached at least a threshold duration after the flow of the first fluid from the first reservoir towards the second reservoir is initiated due to the entropic gradient force.

12. The bio-assembly of claim 11, wherein the threshold duration depends on a size of the fluidic channel and/or a hypertonicity of the second fluid in the second reservoir.

13. The bio-assembly claim 1, wherein the effluent includes cell by-products of cells disposed within the container.

14. The bio-assembly of claim 1, wherein the first fluid includes cell culture media, a therapeutic drug, or a bioactive factor.

15. The bio-assembly of claim 14, wherein the cell culture media include a glucose solution, a serum, an amino acid, an inorganic salt, or a vitamin.

16. The bio-assembly of claim 1, wherein the second fluid includes a hypertonic polymer solution, a stabilizing reagent, or a combination thereof.

17. The bio-assembly of claim 1, wherein the first fluid includes a third fluid and a fourth fluid, and the first reservoir includes a third reservoir configured to receive the third fluid and a fourth reservoir configured to receive the fourth fluid, wherein the third fluid includes one of a cell culture media, a therapeutic drug, or a bioactive factor, and the fourth fluid includes another one of the cell culture media, the therapeutic drug, or the bioactive factor.

18. The bio-assembly of claim 1, wherein the container includes a first container and a second container, the second reservoir includes a third reservoir and a fourth reservoir, the fluidic channel includes a first channel between the first container and the third reservoir and a second channel between the second container and the fourth reservoir.

19. A kit, comprising:
a container defined within a semi-permeable material;
a first reservoir containing a first fluid;
a second reservoir containing a second fluid;
a fluidic channel fluidically connecting the first reservoir and the second reservoir, wherein the first fluid and the second fluid are such that an entropic gradient force causes the first fluid in the first reservoir to flow towards the second reservoir via the fluidic channel; and
a plug positioned between the second reservoir and the fluidic channel, the plug configured to prevent the second fluid from flowing out of the second reservoir via the fluidic channel, wherein:
the container is in fluidic communication with the fluid channel and configured to intake a portion of the first fluid flowing in the fluidic channel from the first reservoir and release cell products produced by cells housed in the container into the first fluid flowing in the fluidic channel towards the second reservoir; and
equilibrium between the first fluid in the first reservoir and the second fluid in the second reservoir is reached at least a threshold duration after the flow of the first fluid from the first reservoir towards the second reservoir is initiated due to the entropic gradient force.

20. The kit of claim 19, wherein the threshold duration depends on a size of the fluidic channel and/or a hypertonicity of the second fluid.
